(19) United States
(12) Patent Application Publication
(10) Pub. No.: US 2005/0276762 A1
(43) Pub. Date: Dec. 15, 2005

(54) Topical compositions containing 5'-adenosine-diphosphate ribose

(76) Inventors: Tapas Das, Worthington, OH (US); Robert A. Shalwitz, Bexley, OH (US)

Correspondence Address: ROSS PRODUCTS DIVISION OF ABBOTT LABORATORIES;DEPARTMENT 108140-DS/1, 625 CLEVELAND AVENUE, COLUMBUS, OH, 43215-1724, US

(21) Appl. No.: 10/868,557

(22) Filed: Jun. 15, 2004

(51) Int. Cl. A61K 7/42

(52) U.S. Cl. 424/59

(57) ABSTRACT

Disclosed are topical skin care compositions and corresponding methods of using those compositions in preventing, retarding, or treating the harmful effects of solar radiation on skin. The compositions comprise 05′-adenosine-diphosphate ribose (ADPR) and a dermatologically acceptable carrier, wherein the compositions are applied topically to the skin prior to, during, or shortly after exposure to the sun. It has been found that 05′-adenosine-diphosphate ribose is unique among many nucleoside derivatives in protecting skin cells from the harmful effects of solar radiation, especially UV radiation.

### TECHNICAL FIELD

The present invention is directed to topical skin care compositions containing 5′-adenosine-diphosphate ribose (ADPR) for preventing, retarding or treating the harmful effects of solar radiation on the skin.

### BACKGROUND OF THE INVENTION

It is well known that prolonged or excessive exposure to sunlight can pose a number of hazards to the skin, most notable of which are photoaging and photocarcinogenesis. Short-term exposure to the sun can result in erythema or sunburn, which primarily results from solar radiation having a wavelength of from about 290 nm to about 320 nm, also referred to as UVB radiation. This type of exposure, especially when prolonged or repeated, can also promote the development of malignant changes in exposed skin cells.

Prolonged exposure to the sun can also result in premature aging of the skin due primarily to UVA radiation at a wavelength of from about 320 nm to about 400 nm. Premature aging of the skin is characterized by wrinkling and pigment changes of the skin, along with other physical changes such as cracking, telangiectasis, solar dermatoses, ecchymoses, and loss of elasticity.

There are many different consumer products available today that provide various degrees of protection from solar radiation. These products often come in the form of topical creams or lotions and contain a chemical or physical sunscreen active in combination with a cosmetically suitable carrier. Chemical sunscreens work by absorbing light or energy, thus potentially shielding skin from incurring damage, whereas the physical sunscreen works by reflecting or scattering away UV radiation from skin.

Examples of common sunscreens include chemical actives such as aminobenzoic acid and derivatives (e.g. para aminobenzoic acid, glyceryl para aminobenzoic acid, padimate 0, Roxadimate,), anthranilates (e.g., menthyl anthranilate), benzophenones (e.g., dioxybenzone, oxybenzone, sulisobenzone), camphor derivatives, (e.g., benzoate-4 methylbenzylidene camphor, mexoryl SX), cinnamates (e.g., octocrylene, octyl methoxycinnamate), dibenzoylmethanes (e.g., avobenzone), salicylates (e.g., homosalate, octyl salicylate, trolamine salicylate), and others (e.g., phenyl benzimidazole). Examples of common physical sunscreens include titanium dioxide, and zinc oxide.

It is also well known, however, that even the most effective topical sunscreens do not provide complete protection from UV radiation. In still allowing some exposure, UV radiation can potentially cause DNA damage within the skin cells by increasing reactive oxygen species (ROS) that facilitate DNA oxidation. The ROS, such as superoxide anion, hydrogen peroxide, and singlet oxygen, can play a critical role in many pathological conditions, including immune suppression, photoaging, and photocarcinogenesis of the skin. Even relatively low doses of UVB can cause DNA mutation leading to tumor initiation, while occasional high doses can result in DNA damage causing apoptotic cell formation (sunburn) and eventually cell deletion.

It has now been found, however, that topical skin care products can be rendered more effective in protecting against UV solar radiation, especially UVB radiation, by adding 5′-adenosine diphosphate ribose (ADPR) to the products. It has been found that this particular compound can protect the skin from solar radiation on a cellular level, even after exposure and generation of reactive oxygen species, unlike many currently available sunscreens. Moreover, ADPR is water-soluble and easily formulated into most topical products, especially those containing an aqueous carrier.

This discovery was made after evaluating several nucleoside derivatives for the protection of cells from the cytotoxic effects from hydrogen peroxide (see FIG. 1). Among the nucleoside derivatives tested, only ADPR provided maximum protection against peroxide mediated cell damage, as well as maximum inhibition against poly (ADP-ribose) polymerase activity, and thus only ADPR could be effectively selected and used from this group of nucleoside derivatives to protect skin cells from solar UVA and UVB radiation. It is not entirely understood why ADPR stood out in this respect among the many nucleoside derivatives tested.

It is therefore an object of the present invention to provide a new ingredient useful for providing skin cells with topical UV protection from solar radiation, and further to provide such an ingredient that can be formulated into a topical skin care product for use alone or in combination with conventional sunscreen actives. It is a further object of the present invention to provide a topical skin care product that can prevent, retard, and treat the adverse effects of solar radiation, and further to provide such a product that works on a cellular level to prevent, reduce, or eliminate reactive oxygen species or free radical mediated cell damage secondary to such exposure.

### SUMMARY OF THE INVENTION

The present invention is directed to topical skin care compositions and corresponding methods of using those compositions in preventing, retarding, and/or treating the harmful effects of solar radiation on skin. The compositions comprise 5′-adenosine-diphosphate ribose (ADPR) and a dermatologically acceptable carrier, wherein the compositions are applied topically to the skin prior to, during, or shortly after exposure to the sun or other similar UV radiation source.

It has been found that ADPR is unique among many nucleoside derivatives in providing skin cells with protection from solar or other forms of UV radiation, especially UVB radiation. Although it is not entirely understood why ADPR stands out in this respect among the many other nucleoside derivatives tested, it was also discovered that ADPR is also unique among many nucleoside derivatives in its ability to inhibit poly (ADP-ribose) polymerase (PARP inhibition), a function that is at least partially responsible for the skin cell protection properties associated with the topical application of ADPR.

### BRIEF DISCRIPTIONS OF DRAWINGS

FIG. 1 is a bar chart representing data from a cytoprotection assay, the cytotoxic effect of H₂O₂ application in accordance with the method described herein, on lung epithelial cells in the presence of either 25 or 50 μg/ml of ADPR or related compounds.

FIG. 2 is a bar chart representing data from an in vitro poly (ADP-ribose) polymerase (PARP) inhibition study showing % PARP inhibition by ADPR and related compounds at 4 mM or 10 mM concentrations.

FIG. 3 is a bar chart representing data from a cytoprotection study in which UVB irradiation at 1, 2, and 4 KJ/m² is applied to immortalized human keratinocytes (HaCat cells) in the presence of 100 μg/ml ADPR.

FIG. 4 is a bar chart representing data from a cytoprotection study in which UVA irradiation at 30, 100, and 300 KJ/m² is applied to immortalized human keratinocytes (HaCat cells) in the presence of 100 μg/ml ADPR.

FIG. 5 is a bar chart representing data from a cytoprotection study in which UVA+UVB irradiation (via solar simulator), as measured by a UV-B probe in terms of UV-B exposure at 150, 450, 600, and 900 J/m², is applied to immortalized human keratinocytes (HaCat cells) in the presence of 100 μg/ml ADPR.

### DETAILED DESCRIPTION OF THE INVENTION

The compositions of the present invention and corresponding methods of application are all directed to topical skin care compositions containing ADPR and a dermatologically acceptable carrier. These and other essential or optional elements or limitations of the compositions and methods of the present invention are described in detail hereinafter.

The terms “topical composition” and “topical skin care composition” are used interchangeably herein, and unless otherwise specified, refer specially to non-oral products that are applied externally to the skin, lips, hair, or nails, and specifically excludes oral compositions and methods of administering oral compositions, or any other non-topical composition or related method of administration, e.g., intravenous, inhalation, nasal, enteral, mouthwash or mouth rinse, etc.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, 5, 6, from 1 to 9, from 3.6 to 4.6, from 3.5 to 9.9, and so forth.

All references to singular characteristics or limitations of the present invention shall include the corresponding plural characteristic or limitation, and vice versa, unless otherwise specified or clearly implied to the contrary by the context in which the references are made.

All combinations of method or process steps as used herein can be performed in any order, unless otherwise specified or clearly implied to the contrary by the context in which the referenced combinations are made.

All percentages, parts and ratios as used herein are by weight of the total composition, unless otherwise specified. All such weights as they pertain to listed ingredients are based on the active level and, therefore, do not include solvents or by-products that may be included in commercially available materials, unless otherwise specified.

The compositions and methods of the present invention can comprise, consist of, or consist essentially of the essential elements and limitations of the invention described herein, as well as any additional or optional ingredients, components, or limitations described herein or otherwise useful in compositions and methods of the general type as described herein.

5′-adenosine Diphosphate Ribose (ADPR)

The topical skin care compositions of the present invention comprise 5′-adenosine diphosphate ribose (ADPR; ADP-ribose; adenosine 5′-(trihydrogen diphosphate),P′→5-ester with D-ribose; adenosine 5′-(trihydrogen pyrophosphate),5′→5-ester with D-ribofuranose; adenosine 5′-diphosphate, D-ribose ester; adenosine 5′-pyrophasphate, 5′→5-ester with D-ribofuranose; ribofuranose, 5-(adenosine 5′-pyrphosphoryl)-D-ribose; adenosine 5′diphosphoribose; adenosine diphosphate ribose; adenosine diphosphoribose; adenosine pyrophosphate-ribose; ribose adenosinediphosphate).

ADPR concentrations suitable for use in the topical skin care compositions are preferably at least about 5 μg/ml (0.0005%), more preferably from about 10 μg/ml (0.001%) to about 500,000 μg/ml (50%), even more preferably from about 10 μg/ml (0.001%) to about 150,000 μg/ml (15%), including from about 10 μg/ml (0.001%) to about 30,000 μg/ml (3%), and also including from about 100 μg/ml (0.01%) to about 10,000 μg/ml (1%).

ADPR is well known in the chemical literature. It is often characterized by the general formula C₁₅H₂₃N₅O₁₄P₂ and includes for example various salts such as those corresponding to the following general structure:

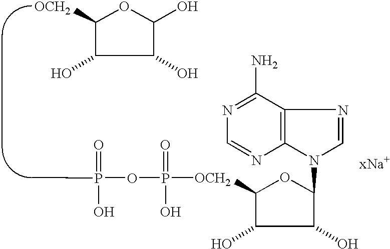

This particular compound, 5-adenosine-diphosphate ribose or ADPR, can be readily prepared by methods well known in the chemical arts. It is also commercially available as a purified raw material, an example of which can be purchased from Sigma-Aldrich Co., St. Louis, Mo., USA.

The ADPR compound for use in the compositions and methods of the present invention includes any known or dermatologically acceptable salt thereof, non-limiting examples of which include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate, undecanoate, or combinations thereof.

The ADPR compound can also include those derivatives in which basic nitrogen-containing groups are quaternized with materials such as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and many others.

Examples of acids which may be employed to form dermatologically acceptable acid addition salts of ADPR include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of the ADPR by reacting a carboxylic acid-containing moiety with a suitable base such as the hydroxide, carbonate or bicarbonate of a pharmaceutically acceptable metal action or with ammonia or an organic primary, secondary or tertiary amine. Non-limiting examples of pharmaceutically acceptable salts include those based on alkali metals or alkaline earth metals such as lithium, sodium, potassium, calcium, magnesium and aluminum salts and the like and nontoxic quaternary ammonia and amine captions including ammonium, tetramethylammonium, tetraethylammonium, methylamine, dimethylamine, trimethylamine, triethylamine, diethylamine, ethylamine and the like. Other representative organic amines useful for the formation of base addition salts include ethyl enediamine, ethanol amine, diethanol amine, piperidine, piperazine, and the like.

Topical Carrier

The topical skin care compositions of the present invention also comprise a dermatologically acceptable carrier suitable for and compatible with the ADPR compound described herein. In this context, a dermatologically acceptable carrier is one that is safe for topical application to the skin, provides consumer acceptable aesthetics when applied topically, and is compatible with ADPR and any other selected actives in the topical skin care composition.

The carrier for use in the topical skin care compositions may be in solid, liquid, or even gaseous form, and may represent all of the topical skin care composition itself other than the ADPR component. The carrier is most typically, however, that portion of the skin care composition other than ADPR and any other skin care or pharmaceutical active, and most typically represents at least about 50%, more typically from about 50% to about 99%, including from about 60% to about 99%, and also including from about 70% to about 98%, and also including from about 80% to about 95%, and also including from about 83% to about 91%, by weight of the topical skin care composition.

The dermatologically acceptable carrier may be, or otherwise may form in combination with the ADPR component, an aqueous or non-aqueous, solid or liquid or gaseous, silicone-containing or non-silicone-containing, single or multi-phase vehicle or product matrix. The carrier may also inherently have or otherwise form with the ADPR component a solid crystalline or non-crystalline matrix, a solution, suspension or emulsion (e.g., oil-in-water, water-in-oil, water-in-oil-in-water, oil-in-water-in-silicone, or other complex emulsion or multiphase system), liquid or solid gel, or any other suitable vehicle or carrier form. Aqueous vehicles are preferred, wherein such aqueous vehicles comprise up to 100% water, including from about 10% to 100%, and also including from about 50% to 100%, and also including from about 60% to about 75%, water by weight of the aqueous vehicle.

Especially useful as carriers are oil-in-water and water-in-oil emulsions, including silicone-in-water and water-in-silicone emulsions. As will be understood by the skilled artisan, a given component or ingredient will distribute primarily into either the water or oil phase, depending upon the water solubility/dispersability of the component in the composition. The ADPR component, for example, most typically and primarily dissolves in or otherwise distributes into an aqueous phase if present in an emulsion system.

The dermatologically acceptable carrier may therefore comprise a lipid or oil phase, as a single or multi-phase system, most typically as part of a stabilized emulsion system. Lipids and oils suitable for use in the carrier may be derived from animals, plants, or petroleum, natural or synthetic. These oil-containing emulsions may further comprise any additional materials suitable for helping to maintain the physical stability of the emulsion system, such as surfactants or emulsifiers well known in the formulation arts, including nonionic, zwitterionic, amphoteric, anionic or cationic emulsifiers, non-limiting examples of which are described in U.S. Pat. No. 3,755,560; U.S. Pat. No. 4,421,769; which descriptions are incorporated by reference herein. Many other suitable emulsifiers are described in McCutcheon's Detergents and Emulsifiers, North American Edition, pages 317-324 (1986).

The dermatologically acceptable carrier may comprise or otherwise form a water-in-silicone emulsion, wherein the emulsion may comprise at least about 1%, including from about 1% to about 60%, also including from about 5% to about 40%, and also including from about 10% to about 20%, by weight of a continuous silicone phase.

Organopolysiloxane oils suitable for use in the carrier may be volatile, non-volatile, or a mixture of volatile and non-volatile silicones. In this context, the term “nonvolatile” refers to those silicones that are solid or liquid under ambient conditions and have a flash point under one (1) atm of or greater than about 100° C. In this context, the term “volatile” refers to all other silicone oils. Suitable organopolysiloxanes can therefore be selected from a wide variety of silicones spanning a broad range of volatilities and viscosities. Non-limiting examples of suitable organopolysiloxane oils include polyalkylsiloxanes, cyclic polyalkylsiloxanes, and polyalkylarylsiloxanes.

Polyalkylsiloxanes suitable for use herein include polyalkylsiloxanes with viscosities of at least about 0.5 centistokes, including from about 0.5 centistokes to about 1,000,000 centistokes at 25° C. Such polyalkylsiloxanes may be represented by the general formula R₃SiO[R₂SiO]ₓ SiR₃ wherein R is an alkyl group having from one (1) to about 30 carbon atoms (including where R is methyl or ethyl or combinations thereof), and x is an integer from 0 to about 10,000, chosen to achieve the desired molecular weight which can range to over about 10,000,000.

Cyclic polyalkylsiloxanes suitable for use herein include those represented by the chemical formula [SiR₂—O]ₙ wherein R is an alkyl group (including methyl or ethyl combinations thereof) and n is an integer from about 3 to about 8, including from about 3 to about 7, and also including from about 4 to about 6. When R is methyl, these materials are typically referred to as cyclomethicones.

Many different organopolysiloxanes may therefore be used as part of the carrier component of the compositions herein, including polyalkylsiloxanes, alkyl substituted dimethicones, cyclomethicones, trimethylsiloxysilicates, dimethiconols, polyalkylaryl siloxanes, and mixtures thereof.

As noted above, the continuous silicone phase when applied to the carrier component may contain one or more non-silicone oils, such as non-silicone containing mineral oil, vegetable oils, synthetic oils, semi synthetic oils, and so forth. A discontinuous silicone phase can likewise comprise similar other oils as well.

The carrier component most typically comprises a continuous or dispersed aqueous phase. As a dispersed phase, the topical skin care composition may comprise up to about 90%, including from about 30% to about 90%, also including from about 50% to about 85%, and also including from about 70% to about 80%, by weight of a dispersed aqueous phase. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The dispersed aqueous phase is a dispersion of small aqueous particles or droplets suspended in and surrounded by the continuous silicone phase described hereinbefore. The aqueous phase can be water, or a combination of water and one or more water soluble or dispersible ingredients. Non limiting examples of such optional ingredients include thickeners, acids, bases, salts, chelants, gums, water-soluble or dispersible alcohols and polyols, buffers, preservatives, additional sunscreening agents, colorings, other polar or semi-polar carriers, and the like.

The skin care compositions of the present invention most typically comprise an aqueous phase, whether continuous or discontinuous, within which most or all of the ADPR dissolves or otherwise partitions or disperses. For water-in-silicone emulsions, the composition may comprise from about 0.1% to about 10% emulsifier, including from about 0.5% to about 7.5%, also including from about 1% to about 5%, of an emulsifier by weight of the composition, to help disperse and suspend the aqueous phase within the continuous silicone phase.

A wide variety of emulsifying agents may be used in the carrier to form, for example, a water-in-silicone emulsion. Known or conventional emulsifying agents can be used in the composition, provided that the selected emulsifying agent is chemically and physically compatible with the essential components of the composition, and provides the desired dispersion characteristics. Suitable emulsifiers include silicone emulsifiers, non-silicone-containing emulsifiers, and mixtures thereof, known by those skilled in the art for use in topical skin care products. These emulsifiers may have an HLB value of or less than about 14, including from about 2 to about 14, and also including from about 4 to about 14. Emulsifiers having an HLB value outside of these ranges can also be used in combination with other emulsifiers to achieve an effective weighted average HLB for the combination that falls within these ranges.

Suitable emulsifiers include silicone emulsifiers, including organically modified organopolysiloxanes, also known to those skilled in the art as silicone surfactants. Useful silicone emulsifiers include dimethicone copolyols. These materials are polydimethylsiloxanes modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, i.e., compounds that contain C2-C30 pendant side chains. Still other useful dimethicone copolyols include materials having various cationic, anionic, amphoteric, and zwitterionic pendant moieties.

Among the many non-silicone-containing emulsifiers useful herein are various non-ionic and anionic emulsifying agents such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof. Other suitable emulsifiers are described, for example, in McCutcheon's, Detergents and Emulsifiers, North American Edition (1986), published by Allured Publishing Corporation; U.S. Pat. No. 5,011,681; U.S. Pat. No. 4,421,769; and U.S. Pat. No. 3,755,560, which descriptions are incorporated by reference herein. Non-limiting examples of such non-silicon-containing emulsifiers include: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, steareth-20, ceteareth-20, PPG-2 methyl glucose ether distearate, ceteth-10, diethanolamine cetyl phosphate, glyceryl stearate, PEG-100 stearate, and mixtures thereof.

The dermatologically acceptable carrier may also be or otherwise form in combination with the ADPR component an oil-in-water emulsion having a continuous aqueous phase and a hydrophobic, water-insoluble phase (“oil phase”) dispersed therein. Non-limiting examples of suitable carriers comprising oil-in-water emulsions are described in U.S. Pat. No. 5,073,371 and U.S. Pat. No. 5,073,372, which descriptions are incorporated by reference herein.

An oil-in-water carrier for use herein may further comprise a structuring agent to assist in the formation of a liquid crystalline gel network structure, concentrations of which may range from about 0.5% to about 20%, including from about 1% to about 10%, and also including from about 1% to about 5%, by weight of the topical skin care composition, of a structuring agent. Non limiting examples of such structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

Additional Sunscreen Active

The topical skin care compositions of the present invention may further comprise any additional sunscreen active that is known for or otherwise effective in providing protection from solar radiation when applied topically to the skin. These sunscreen actives must therefore be safe for topical application to the skin and must also be compatible with the other selected ingredients in the composition. The optional sunscreen active is used in combination with ADPR and the dermatologically acceptable carrier.

Sunscreen actives suitable for use herein may be used at any concentration that is safe for topical application to the skin, but most typically range up to about 20%, including from about 1% to about 10%, including from about 2% to about 8%, by weight of the topical skin care composition. Exact amounts vary depending upon factors such as the particular sunscreen chosen and the desired Sun Protection Factor (SPF) desired.

Sunscreen actives suitable for use herein include both chemical absorbers and physical blockers as defined by their respective mechanisms of action. Chemical sunscreens are generally aromatic compounds conjugated with a carbonyl group that absorb high intensity UV rays with excitation to a higher energy state, non-limiting examples of which include chemical sunscreens such as aminobenzoic acid and derivatives (e.g. para aminobenzoic acid, glyceryl para aminobenzoic acid, padimate O, Roxadimate,), anthranilates (e.g., menthyl anthranilate), benzophenones (e.g., dioxybenzone, oxybenzone, sulisobenzone), camphor derivatives, (e.g., benzoate-4 methylbenzylidene camphor, mexoryl SX), cinnamates (e.g., octocrylene, octyl methoxycinnamate), dibenzoylmethanes (e.g., avobenzone), salicylates (e.g., homosalate, octyl salicylate, trolamine salicylate), and others (e.g., phenyl benzimidazole).

Physical sunscreens or blockers suitable for use herein include those that reflect or scatter UV radiation and are most typically in the form of inorganic particulates, non-limiting examples of which include titanium dioxide, zinc oxide, or combinations thereof.

Especially useful and commonly used sunscreen actives include 4,4′-t-butylmethoxydibenzoylmethane, 2-ethylhexyl-p-methoxycinnamate, phenyl benzimidazole sulfonic acid, octocrylene, zinc oxide, and titanium dioxide, and mixtures thereof.

Optional Ingredients

The topical skin care compositions of the present invention may further comprise any of a variety of other ingredients, active or inert, which are known or otherwise suitable for use in topical skin care products. Such optional ingredients should be safe for topical application to the skin and compatible with any other selected ingredients in the composition.

Many different optional ingredients are described in The CTFA Cosmetic Ingredient Handbook, Second Edition (1992), including a wide variety of cosmetic and pharmaceutical ingredients commonly used in the skin care industry, which are also suitable for use in the compositions of the present invention. Non-limiting examples of some suitable optional ingredients include abrasives, absorbents, aesthetic components such as fragrances, pigments, colorings/colorants, essential oils, skin sensates and astringents e.g., clove oil, menthol, camphor, eucalyptus oil, eugenol, menthyl lactate, witch hazel distillate), anti-acne agents, anti-caking agents, antifoaming agents, antimicrobial agents, additional antioxidants, binders, biological additives, buffering agents, bulking agents, chelating agents, cosmetic biocides, denaturants, external analgesics, film forming polymers, opacifying agents, pH adjusters, propellants, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives such as ethyl panthenol, aloe vera, pantothenic acid and its derivatives, allantoin, bisabolol, and dipotassium glycyrrhizinate), thickeners, and vitamins and derivatives thereof, and combinations thereof.

The topical skin care compositions of the present invention may further comprise an additional skin care active including desquamatory actives, anti-acne actives, wrinkle repair actives, vitamin B₃ compounds, retinoids, chelators, anti-inflammatory agents, topical anesthetics, tanning actives, skin lightening agents, anti-cellulite agents, flavonoids, antimicrobial actives, antifungal actives, sunscreen actives, skin conditioning agents, and combinations thereof. Such additional skin care actives when formulated into the topical skin care composition most typically represent from about 0.001% to about 30%, including from about 0.01% to about 10%, also including from about 0.1% to about 7%, and also including from about 1% to about 5%, by weight of the topical skin care composition.

Other suitable skin care actives include one or more vitamin B₆ compounds such as pyridoxine, esters of pyridoxine (e.g., pyridoxine tripalmitate), amines of pyridoxine (e.g., pyridoxamine), salts of pyridoxine (e.g., pyridoxine HCl) and derivatives thereof, including pyridoxamine, pyridoxal, pyridoxal phosphate, and pyridoxic acid. Especially useful are pyridoxine, esters of pyridoxine and salts of pyridoxine.

Pyridoxine HCl is well known for use in topical skin care compositions and is also suitable for use herein. These vitamin B₆ compounds are most typically used in the topical skin care compositions at concentrations ranging from about 0.0001% to about 25% by weight of the composition, more typically from about 0.001% to about 10%, including from about 0.01% to about 5%, and also including from about 0.1% to about 2.5%, by weight of the skin care composition. These actives are especially useful in reducing the appearance of wrinkles and other age-related skin imperfections.

Non-limiting examples suitable anti-acne actives for use herein include resorcinol, sulfur, salicylic acid, erythromycin, zinc, and other actives such as those described in U.S. Pat. No. 5,607,980, which description is incorporated herein by reference.

Non-limiting examples of suitable anti-wrinkle/anti-atrophy actives suitable for use herein include sulfur-containing D and L amino acids and their derivatives and salts, particularly the N-acetyl derivatives such as N-acetyl-L-cysteine; thiols, e.g. ethane thiol; hydroxy acids (e.g., salicylic acid, glycolic acid), keto acids (e.g., pyruvic acid), ascorbic acid (vitamin C), phytic acid, lipoic acid; lysophosphatidic acid, skin peel agents (e.g., phenol and the like), flavonoids (e.g., flavanones, chalcones, isoflavones, flavones, etc.), stilbenes, cinnamates, resveratrol, kinetin, zeatin, dimethylaminoethanol, peptides from natural sources (e.g., soy peptides), salts of sugar acids (e.g., Mn gluconate), terpene alcohols (e.g., farnesol), peptides, vitamin B₃ compounds and retinoids which enhance the keratinous tissue appearance benefits of the present invention, especially in regulating keratinous tissue condition, e.g., skin condition, and other vitamin B compounds (e.g., thiamine (vitamin B₁), pantothenic acid (vitamin B₅), camitine, riboflavin (vitamin B₂), cyanocobalamine (vitamin B₁₂), pangamic acid or diisopropylamine dichloroacetate, and their derivatives and salts (e.g., HCl salts or calcium salts)).

Vitamin B₃ compounds are especially useful in the skin care compositions of the present invention for regulating skin condition, concentrations of which may range from about 0.01% to about 50%, including from about 0.1% to about 10%, also including from about 0.5% to about 10%, also including from about 1% to about 5%, and also including from about 2% to about 5%, by weight of the topical skin care composition. These vitamin B₃ and related compounds include niacinamide, nicotinic acid, nicotinyl alcohol, tocopheryl nicotinate, nictinyl amino acids, nicotinyl alcohol esters of carboxylic acids, nicotinic acid N-oxid and niacinamide N-oxide, and derivatives and/or salts thereof, and combinations thereof. Niacinamide is preferred.

Optional retinoids for use herein include all natural or synthetic analogs of Vitamin A or retinol-like compounds that possess the biological activity of Vitamin A in the skin as well as the geometric isomers and stereoisomers of these compounds. The retinoid is preferably retinol, retinol esters (e.g., C₂-C₂₂ alkyl esters (saturated or unsaturated alkyl chains) of retinol, including retinyl palmitate, retinyl acetate, retinyl propionate), retinal, and/or retinoic acid (including all-trans retinoic acid and/or 13-cis-retinoic acid). Other suitable retinoids include tocopheryl-retinoate [tocopherol ester of retinoic acid (trans- or cis-), adapalene {6-[3-(1-adamantyl)-4-methoxyphenyl]-2-naphthoic acid}, and tazarotene (ethyl 6-[2-(4,4-dimethylthioch-roman-6-yl)-ethynyl]nicotinate). Retinoid concentrations in the topical compositions may range from about 0.005% to or about 2%, including from about 0.01% to about 2%, by weight of the composition. Retinol concentrations range from about 0.01% to about 0.15%; retinol ester concentrations range from or about 0.01% to about 2%; retinoic acids concentrations range from about 0.01% to about 0.25%; tocopheryl-retinoate, adapalene, and tazarotene concentrations range from about 0.01% to about 2%, all by weight of the skin care composition.

Optional anti-oxidants and radical scavengers for use in the topical skin care compositions may be formulated at concentrations of from about 0.001% to about 10%, including from about 0.1% to about 5%, by weight of the topical skin care composition. Non-limiting examples of such ingredients include ascorbic acid and its salts, ascorbyl esters of fatty acids, ascorbic acid derivatives (e.g., magnesium ascorbyl phosphate), tocopherol (vitamin E), tocopherol sorbate, tocopherol acetate, other esters of tocopherol, butylated hydroxy benzoic acids and their salts, 6-hydroxy-2,5,7,8-tetramethylchrom-an-2-carboxylic acid (commercially available under the tradename Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulthydryl compounds (e.g., glutathione), dihydroxy fulmaric acid and its salts, lycine pidolate, arginine pilolate, nordihydroguaiaretic acid, bioflavonoids, lysine, methionine, proline, superoxide dismutase, silymarin, tea extracts, grape skin/seed extracts, melanin, and rosemary extracts.

Optional chelators for use in the topical skin care compositions include any active agent capable of removing a metal ion from a system by forming a complex so that the metal ion cannot readily participate in or catalyze chemical reactions. The inclusion of a chelating agent is especially useful for providing protection against UV radiation that can contribute to excessive scaling or skin texture changes and against other environmental agents, which can cause skin damage. Concentrations of the optional chelating agent may range from about 0.1% to about 10%, including from about 1% to about 5%, by weight of the composition. Non-limiting examples of such ingredients include furildioxime and other chelators such as those described in U.S. Pat. No. 5,487,884, which description is incorporated herein by reference.

Optional flavonoids for use in the topical skin care compositions include those described in U.S. Pat. Nos. 5,686,082 and 5,686,367, both descriptions of which are incorporated herein by reference. Suitable flavonoids include unsubstituted flavanones, mono-substituted flavanones, and mixtures thereof, chalcones selected from the group consisting of unsubstituted chalcones, mono-substituted chalcones, di-substituted chalcones, tri-substituted chalcones, and mixtures thereof, flavones selected from the group consisting of unsubstituted flavones, mono-substituted flavones, di-substituted flavones, and mixtures thereof, one or more isoflavones; coumarins selected from the group consisting of unsubstituted coumarins, mono-substituted coumarins, di-substituted coumarins, and mixtures thereof, chromones selected from the group consisting of unsubstituted chromones, mono-substituted chromones, di-substituted chromones, and mixtures thereof, one or more dicoumarols; one or more chromanones; one or more chromanols; isomers (e.g., cis/trans isomers) thereof, and mixtures thereof.

By the term “substituted” as used herein means flavonoids wherein one or more hydrogen atom of the flavonoid has been independently replaced with hydroxyl, C1-C8 alkyl, C1-C4 alkoxyl, O-glycoside, and the like or a mixture of these substituents. Examples of suitable flavonoids include, but are not limited to, unsubstituted flavanone, mono-hydroxy flavanones (e.g., 2′-hydroxy flavanone, 6-hydroxy flavanone, 7-hydroxy flavanone, etc.), mono-alkoxy flavanones (e.g., 5-methoxy flavanone, 6-methoxy flavanone, 7-methoxy flavanone, 4′-methoxy flavanone, etc.), unsubstituted chalcone (especially unsubstituted trans-chalcone), mono-hydroxy chalcones (e.g., 2′-hydroxy chalcone, 4′-hydroxy chalcone, etc.), di-hydroxy chalcones (e.g., 2′,4-dihydroxy chalcone, 2′,4′-dihydroxy chalcone, 2,2′-dihydroxy chalcone, 2′,3-dihydroxy chalcone, 2′,5′-dihydroxy chalcone, etc.), and tri-hydroxy chalcones (e.g., 2′,3′,4′-trihydroxy chalcone, 4,2′,4′-trihydroxy chalcone, 2,2′,4′-trihydroxy chalcone, etc.), unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones (a mixture extracted from soy), unsubstituted coumarin, 4-hydroxy coumarin, 7-hydroxy coumarin, 6-hydroxy-4-methyl counarin, unsubstituted chromone, 3-formyl chromone, 3-formyl-6-isopropyl chromone, unsubstituted dicoumarol, unsubstituted chromanone, unsubstituted chromanol, and mixtures thereof.

Optional anti-inflammatory agents for use in the topical skin care composition may be formulated at concentrations ranging from about 0.1% to about 10%, including from about 0.5% to about 5%, by weight of the composition. Non-limiting examples of steroidal anti-inflammatory agents, include hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Optional nonsteroidal anti-inflammatory agents suitable for use in the topical skin care compositions include oxicams (e.g., piroxicam, isoxicam, tenoxicam, sudoxicam), salicylates (e.g., aspirin, disalcid, benorylate, trilisate, safapryn, solprin, diflunisal, and fendosal), acetic acid derivatives (e.g., diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac), fenamates (e.g, mefenamic, meclofenamic, flufenamic, niflumic, and tolfenamic acids), propionic acid derivatives (e.g, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic); and pyrazoles (e.g., phenylbutazone, oxyphenbutazone, feprazone, azapropazone, trimethazone).

Still other anti-inflammatory agents suitable for use herein include allantoin and compounds of the Licorice (the plant genus/species Glvcvrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, preferably C.sub.10-C.sub.24, more preferably C16-C24, specific examples of which include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrr-hetinate.

Other optional ingredients for use in the topical skin care compositions include topical anesthetics, non-limiting examples of which include benzocaine, lidocaine, bupivacaine, chlorprocaine, dibucaine, etidocaine, mepivacaine, tetracaine, dyclonine, hexylcaine, procaine, cocaine, ketarnne, pramoxine, phenol, and pharmaceutically acceptable salts thereof.

Still other optional ingredients include tanning actives at concentrations ranging from about 0.1% to about 20%, including from about 2% to about 7%, and also including from about 3% to about 6%, by weight of the topical skin care composition. Non-limiting examples of such actives include dihydroxyacetone.

Other optional ingredients include skin lightening agents, concentrations of which my range from about 0.1% to about 10%, including from about 0.2% to about 5%, and also including from about 0.5% to about 2%, by weight of the topical skin care composition. Non-limiting examples of suitable skin lightening agents including kojic acid, arbutin, tranexamic acid, ascorbic acid and derivatives thereof, e.g., magnesium ascorbyl phosphate or sodium ascorbyl phosphate or other salts of ascorbyl phosphate.

The compositions of the present invention may further comprise an antimicrobial or antifungal active. A safe and effective amount of an antimicrobial or antifungal active may be added to the compositions, including from about 0.001% to about 10%, also including from about 0.01% to about 5%, and also including from about 0.05% to about 2%, by weight of the skin care composition.

Non-limiting examples of antimicrobial and antifungal actives include B-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione, clotrimazole, and combinations thereof.

Examples of optional actives especially useful herein include those selected from the group consisting of salicylic acid, benzoyl peroxide, 3-hydroxy benzoic acid, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, phytic acid, N-acetyl-L-cysteine, lipoic acid, azelaic acid, arachidonic acid, benzoylperoxide, tetracycline, ibuprofen, naproxen, hydrocortisone, acetaminophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, and combinations thereof.

The topical skin care compositions of the present invention may further comprise a conditioning agent, including humectants, moisturizers, skin conditioners, or combinations thereof, concentrations of which most typically range from at least about 0.01%, preferably from about 0.01% to about 20%, including from about 0.1% to about 10%, and also including from about 0.5% to about 7%, by weight of the topical skin care composition. Non-limiting examples of such conditioning agents include guanidine; urea; glycolic acid and glycolate salts (e.g. ammonium and quaternary alkyl ammonium); lactic acid and lactate salts (e.g., ammonium and quaternary alkyl ammonium); aloe vera in any of its variety of forms (e.g., aloe vera gel); polyhydroxy compounds such as sorbitol, mannitol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fructose, sucrose, etc.); hyaluronic acid; lactamide monoethanolamine; acetamide monoethanolamine; and mixtures thereof. Glycerol is especially useful herein.

The topical skin care compositions of the present invention may further comprise a thickening agent, concentrations of which most typically range from at least about 0.1%, including from about 0.1% to about 5%, more typically about 0.1% to about 3%, and also including from about 0.25% to about 2%, by weight of the topical skin care composition. Non-limiting examples of suitable thickening agents include carboxylic acid polymers, crosslinked polyacrylate polymers, polyacrylamide polymers, and mixtures thereof.

### Product Form

The topical skin care compositions of the present invention may be prepared or otherwise applied topically to the skin in any known or otherwise suitable product form, such as lotions, creams, ointments, patches, suspensions, pump or aerosol sprays, solutions, bars, gels, wipes, and so forth. The topical skin care compositions may include any solid, semi-solid, or liquid form, including variations thereof such as pressurized aerosols, powders, impregnated or absorbed wipes or other solid substrate matrix, and so forth.

The topical skin care compositions include lotions and creams, which most typically further comprise from about 2% to about 50% of an emollient, by weight of the topical skin care composition. In this context, an emollient refers to a material useful for the prevention or relief of dryness and related protection of the skin. A wide variety of suitable emollients are known and may be used herein, such as those described in Sagarin, Cosmetics, Science and Technology, 2nd Edition, Vol. 1, pp. 32-43 (1972). Glycerin, for example, is a commonly used emollient, concentrations of which most typically range from about 0.001% to about 20%, more typically from about 0.01% to about 10%, and even more typically from about 0.1% to about 7%, by weight of the topical skin care composition.

Lotions most typically comprise from about 1% to about 20%, including from about 5% to about 10%, of an emollient; from about 50% to about 90%, including from about 60% to about 80%, water by weight of the topical skin care composition. Creams most typically comprise from about 5% to about 50%, including from about 10% to about 20%, of an emollient; from about 45% to about 85%, including from about 50% to about 75%, water, by weight of the skin care composition.

Ointment embodiments of the present invention most typically comprise a simple carrier base of animal or vegetable oils or semi-solid hydrocarbons (oleaginous); absorption ointment bases which absorb water to form emulsions; or water soluble carriers, e.g., a water soluble solution carrier. Ointments may further comprise a thickening agent, such as described in Sagarin, Cosmetics, Science and Technology, 2nd Edition, Vol. 1, pp. 72-73 (1972), and/or an emollient.

Other suitable product forms include cleansing compositions for the hair, face, or other area of the skin, which comprise a suitable skin cleansing surfactant. The cleansing composition may include toilet bars, liquid hand cleansers, hair or body shampoos, bath gels, hair conditioners, hair tonics, pastes, or mousses, and so forth. These compositions preferably contain a delivery system adequate to deposit ADPR and any other skin active agent onto the intended area of application on the skin, hair or scalp. Non-limiting examples of some such delivery systems are described in U.S. Pat. No. 4,835,148, which description is incorporated herein by reference.

The topical skin care compositions of the present invention may also be formulated in the form of color or other cosmetics, non-limiting examples of which include foundations, lipsticks, rouges, mascaras, and the like. Such cosmetic products may include conventional ingredients such as oils, colorants, pigments, emollients, fragrances, waxes, stabilizers, and the like.

The topical skin care compositions of the present invention may also be formulated as an insect repellant, lip balm, fine perfume or aftershave, hair spray, hair mousse, hair conditioner, scalp conditioner or treatment, or any other product form that can be applied topically to the skin, wherein the ADPR provides the topical composition with secondary or additional UV protection benefits described herein.

### Method of Making

The topical skin care compositions of the present invention may be prepared by any conventional or otherwise known method suitable for preparing or manufacturing the selected product form. Such methods can vary significantly depending upon the product form selected (e.g, aqueous solution, cream, lotion, solid wax stick, aerosol or pump spray, etc.)

For many cream or lotion embodiments, for example, the ingredients can often be combined and mixed together in one or more steps to a relatively uniform state, with or without heating, cooling, application of vacuum, and the like.

For those embodiments comprising water or a separate aqueous phase, the ADPR component may be dissolved or dispersed in the water component or aqueous phase, and thereafter formulated with the remaining product ingredients in a conventional manner for that particular product form and the selected ingredients therein.

Specific non-limiting examples of some commonly known or otherwise applied methods of making topical skin care products, which can be applied to the formulation of the topical skin care products of the present invention, are described hereinafter in association with several exemplified skin care formulations.

### Methods of Use

The topical skin care compositions of the present invention are useful in protecting the skin from, or treating it in response to, the harmful effects UV radiation sources such as solar radiation or sunlight as well as other harmful UV radiation sources. The present invention is therefore also directed to a method of preventing, retarding, and/or treating the harmful effects of UV radiation, including UVA, UVB, and combinations thereof, most typically radiation from the sun, said method comprising the step of topically applying to skin in need of such prevention or treatment a skin care composition comprising 05′-adenosine-diphosphate ribose and a suitable topical carrier.

The topical composition of the present invention can be applied prior to, during, or shortly after exposure to UV radiation such as solar radiation or sunlight. In this context, the “prior to” means within 24 hours of such exposure, including within 0 to 8 hours, including within about 1 hour, including immediately prior to such exposure. The term “shortly after exposure” as used herein means that the topical skin care composition may be applied with 0 to 8 hours, including from 0 to 1 hour, and also including immediately after, exposure to UV radiation such as solar radiation or sunlight. The topical composition is preferably applied as a leave-on composition, wherein the composition is applied to and left on the skin for up to 24 hours or more, including up to 12 hours, including up to 8 hours, and also including from 0.5 to 2 hours, following application.

The methods of the present invention may be directed to any external area of the skin exposed to solar or UV irradiation, including those areas covering the face or lips, hair or scalp, neck, front or back or sides of the torso, arms, hands, legs, feet, fingernails, toenails, and so forth. The topical skin care composition can be applied with fingers or hands or by using an appropriate implement such as a woven or non-woven fabric or other solid matrix or directly through a product form applicator such as an aerosol or pump spray, roller-ball applicator, and so forth.

The amount of product to be applied depends upon a number of commonly balanced variables such as the selected product form, site of application, other secondary or primary uses for the product form other than mere UV radiation protection, active concentration including ADPR concentration in the selected product form, and so forth. These compositions are preferably leave-on formulations applied to the skin and allowed to remain for prolonged periods, except that the compositions can also be formulated as a rinse-off formulation such as a hand cleanser, hair shampoo, body cleanser, face cleanser, and so forth. For rinse-off compositions, it is preferred that the composition is formulated so that at least some ADPR remains on the applied area after the product is rinsed or wiped away. In this type of application, it is sometimes helpful to include deposition aids to enhance delivery and retention of the ADPR active to the applied areas of the skin.

The present invention may also be directed to the application of the compositions herein to hair to provide the hair with improved UV protection. Such a method may also further provide additional UV protection to the scalp as well. Such embodied methods are preferably applied from a rinse-off shampoo or other topical cleanser, although it is understood that a leave-on application to the hair or scalp would also be effective.

The methods of the present invention therefore include the topical application of the compositions of the present invention to prevent or otherwise treat photodamaged skin, wherein the photodamaged skin results from exposure to UV radiation sources such as sunlight, and includes the resulting protection against or treatment for UV radiation induced skin conditions such as photoaging, edema, lympocytic and/or neutrophilic infiltration of the dermis, vasodilation, and dyskeratotic keratinocytes and spongiosis of the epidermis. The methods are preferably directed to the long term prevention of skin atrophy or wrinkles, skin cancer, or combinations thereof. The methods are also directed to the short term prevention of acute photo damage often referred to as “sunburn”.

The methods of the present invention may also be directed to preventing, retarding, or treating the skin, wherein the source of UV solar radiation is an artificial equivalent such as that associated with tanning booths, tanning beds, tanning tables, tanning lights, and so forth.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Example 1

Examples 1.1-1.7 illustrate topical skin cream embodiments of the present invention, including a method of topically applying the cream to prevent or treat the harmful effects of UV solar or sunlight radiation on the skin. Ingredients to formulate each of the skin cream compositions are listed in the following table. Each ingredient amount listed in the table is in kg, unless otherwise specified.

Example 1—Topical Skin Care Creams

| | Ex. | Ex. | Ex. | Ex. | Ex. | Ex. | Ex. |
|---|---|---|---|---|---|---|---|
| Ingredients | 1.1 | 1.2 | 1.3 | 1.4 | 1.5 | 1.6 | 1.7 |
| Phase A Ingredients | | | | | | | |
| Water USP | 57.3 | 57.3 | 57.3 | 57.3 | 57.3 | 57.3 | 57.3 |
| Disodium EDTA | 0.13 | 0.13 | 0.13 | 0.13 | 0.13 | 0.13 | 0.13 |
| Methyl paraben | 0.25 | 0.25 | 0.25 | 0.25 | 0.25 | 0.25 | 0.25 |
| Glycerin | 3.00 | 3.00 | 3.00 | 3.00 | 3.00 | 3.00 | 3.00 |
| Zinc citrate | 1.00 | 1.00 | 1.00 | 1.00 | 1.00 | 1.00 | 1.00 |
| ADPR | 0.0005 | 0.001 | 0.01 | 0.1 | 1.0 | 3.0 | 15.0 |
| Phase B Ingredients | | | | | | | |
| Cetyl Alcohol | 0.56 | 0.56 | 0.56 | 0.56 | 0.56 | 0.56 | 0.56 |
| Stearyl alcohol | 2.03 | 2.03 | 2.03 | 2.03 | 2.03 | 2.03 | 2.03 |
| Steareth-21 (Brij 721) | 0.37 | 0.37 | 0.37 | 0.37 | 0.37 | 0.37 | 0.37 |
| Steareth-2 (Brij 72) | 1.10 | 1.10 | 1.10 | 1.10 | 1.10 | 1.10 | 1.10 |
| Distearyldimonium Cl | 0.95 | 0.95 | 0.95 | 0.95 | 0.95 | 0.95 | 0.95 |
| Propyl paraben | 0.10 | 0.10 | 0.10 | 0.10 | 0.10 | 0.10 | 0.10 |
| Propylene glycol-15 stearyl ether | 3.25 | 3.25 | 3.25 | 3.25 | 3.25 | 3.25 | 3.25 |
| Phase C Ingredients | | | | | | | |
| Propylene glycol-15 stearyl ether | 2.17 | 2.17 | 2.17 | 2.17 | 2.17 | 2.17 | 2.17 |
| Titanium dioxide | 0.75 | — | — | — | — | — | — |
| Phase D | | | | | | | |
| Niacinamide | 3.00 | — | — | — | — | 2.0 | — |
| Salicylic acid | — | 1.5 | — | — | — | — | — |
| Pantothenic acid | — | — | 2.0 | — | — | — | — |
| Pyridoxine HCL | — | — | — | 1.0 | 5.0 | — | — |
| Citric acid | 0.19 | 0.19 | 0.19 | 0.19 | 0.19 | 0.19 | 0.19 |
| Water USP | 17.00 | 17.00 | 17.00 | 17.00 | 17.00 | 17.00 | 17.00 |
| Na OH 50% solution | 0.94 | 0.94 | 0.94 | 0.94 | 0.94 | 0.94 | 0.94 |
| Phase E Ingredients | | | | | | | |
| Benzyl alcohol | 0.50 | 0.50 | 0.50 | 0.50 | 0.50 | 0.50 | 0.50 |
| Cyclomethicone dimethiconol 50/50 | 0.75 | 0.75 | 0.75 | 0.75 | 0.75 | 0.75 | 0.75 |
| blend | | | | | | | |
| Dimethicone 10 cst | 1.00 | 1.00 | 1.00 | 1.00 | 1.00 | 1.00 | 1.00 |
| Polyethylene low density beads | — | 1.00 | 1.00 | 1.00 | 1.00 | 1.00 | 1.00 |
| Phase F ingredients | | | | | | | |
| Fragrance | — | 0.10 | 0.10 | 0.10 | 0.10 | 0.10 | 0.10 |
| Phase G ingredients | | | | | | | |
| NaOH 50% solution | | 0.33 | 0.33 | 0.33 | 0.33 | 0.33 | 0.33 |

The exemplified skin creams are prepared by conventional methods by formulating and combining the above-described Phase A-G ingredients. Initially, the Phase A ingredients are combined and mixed together at 70-80° C. In a separate mixer, the Phase B ingredients are combined, mixed, heated and melted together, while in another mixer the Phase C ingredients are combined and milled together to obtain an acceptably smooth mixture (e.g., using a Tekmar T50 Mill). The Phase B and C ingredients and then combined and mixed together, with the resulting B-C combination subsequently combined and mixed with the Phase A ingredients. The ABC combination is cooled with a cold water bath and mill within continued stirring. The combination is removed from the bath, with continued stirring, once the temperature reaches 40° C.

Separately, the Phase D ingredients are combined and mixed together until dissolved, and then subsequently combined with the ABC combination described above. Separately, the Phase E ingredients are combined and mixed together until smooth and continuous, after which the mixture is added to the ABCD combination. Fragrance is added to the ABCDE combination followed by NaOH addition. If necessary, the pH is adjusted to 5.5.

The resulting skin creams are applied topically, typically once or twice daily, to the skin to reduce fine lines and wrinkles and improve skin surface texture, and thereafter also provides protection from exposure to solar UV radiation. The Example 2.2 lotion is applied topically as needed to the skin prior to, during, and/or after exposure to solar radiation or sunlight, and thereafter retards, prevents, or treats the skin from the harmful effects of such solar radiation or sunlight exposure.

Each of the resulting skin cream compositions is applied topically as needed for the desired UV solar protection, and for those embodiments also containing additional skin active agents, the creams are more typically applied once or twice daily to the skin to reduce fine lines and wrinkles or otherwise improve skin surface texture. All of the exemplified creams may be applied topically as needed to the skin prior to, during, and/or after exposure to solar radiation or sunlight, and thereafter retards, prevents, or treats the skin from the harmful effects of solar radiation or sunlight exposure.

### Example 2

The following Examples 2.1-2.7 illustrate topical lotion or emulsion embodiments of the present invention, including a method of topically applying the lotions to prevent, retard, and/or treat the harmful effects of UV solar or sunlight radiation on the skin. Ingredients to formulate each topical lotion or emulsion are listed in the following table. All ingredient amounts, unless otherwise specified, are weight percentages based upon the total weight of the skin cream composition.

Example 2—Topical Skin Care Lotions

| | Ex. | Ex. | Ex. | Ex. | Ex. | Ex. | Ex. | Ex. |
|---|---|---|---|---|---|---|---|---|
| Ingredient | 2.1 | 2.2 | 2.3 | 2.4 | 2.5 | 2.6 | 2.7 | 2.7 |
| Silicone fluid (Dow | 15 | 15 | 15 | 15 | 15 | 15 | 15 | 15 |
| Corning DC 345) | | | | | | | | |
| Silicone fluid (Dow | 2.5 | 2.5 | 2.5 | 2.5 | 2.5 | 2.5 | 2.5 | 2.5 |
| Corning DC 3225 C | | | | | | | | |
| Silicone fluid | 2.5 | 2.5 | 2.5 | 2.5 | 2.5 | 2.5 | 2.5 | 2.5 |
| (Goldschmidt Abil | | | | | | | | |
| We09) | | | | | | | | |
| Niacinamide | 3.0 | — | — | — | — | — | — | — |
| Salicylic acid | — | 1.5 | — | — | — | — | — | — |
| Chemical sunscreen | — | — | 2.0 | — | — | — | — | — |
| active¹ | | | | | | | | |
| Physical sunscreen | — | — | — | 3.0 | — | — | — | — |
| active² | | | | | | | | |
| Pantothenic acid | — | — | — | — | 2.0 | — | — | — |
| Tocopherol acetate | — | — | — | — | — | 0.5 | — | — |
| Pyridoxine HCL | — | — | — | — | — | — | 1.0 | — |
| Benzyl alcohol | 0.3 | 0.3 | 0.3 | 0.3 | 0.3 | 0.3 | 0.3 | 0.3 |
| Methyl paraben | 0.2 | 0.2 | 0.2 | 0.2 | 0.2 | 0.2 | 0.2 | 0.2 |
| Glycerin | 3.0 | 3.0 | 3.0 | 3.0 | 3.0 | 3.0 | 3.0 | 3.0 |
| Tetrasodium EDTA | 0.1 | 0.1 | 0.1 | 0.1 | 0.1 | 0.1 | 0.1 | 0.1 |
| ADPR | 0.0005 | 0.001 | 0.01 | 0.1 | 1.0 | 3.0 | 15.0 | 0.0005 |
| Benzyl alcohol | 0.3 | 0.3 | 0.3 | 0.3 | 0.3 | 0.3 | 0.3 | 0.3 |
| Water | q.s. | q.s. | q.s. | q.s. | q.s. | q.s. | q.s. | q.s. |
| Total | 100 | 100 | 100 | 100 | 100 | 100 | 100 | 100 |

¹Chemical susnscreen actives: para aminobenzoic acid, glyceryl para aminobenzoic acid, padimate O, Roxadimate, menthyl anthranilate, dioxybenzone, oxybenzone, sulisobenzone, benzoate-4 methylbenzylidene camphor, mexoryl SX, octocrylene, octyl methoxycinnamate, avobenzone, homosalate, octyl salicylate, trolamine salicylate, phenyl benzimidazole, combinations thereof.
²Physical sunscreen actives: titanium dioxide, zinc oxide, and combinations thereof.

The exemplified lotions or emulsions are prepared by conventional methods. For example, an aqueous phase is prepared initially by combining and mixing in a suitable vessel charged with water the glycerin component followed by the ADPR and the skin active agent (e.g., niacinamide, salicylic acid, sunscreen active, pantothenic acid, pyridoxine HCL, tocopherol acetate) and to that resulting mixture is added with mixing the methyl paraben dissolved in the benzyl alcohol. EDTA is then combined and mixed with the resulting combination.

A silicone oil phase is prepared in a separate suitable vessel by adding and stirring together the silicone fluids. The aqueous phase is then slowly combined and mixed with the silicone phase to form a lotion or emulsion embodiment of the present invention.

The resulting lotion or emulsion compositions are applied topically as needed, most typically once or twice daily depending upon the skin active agent in the formulation and its intended purpose (e.g, to reduce fine lines and wrinkles and improve skin surface texture), and thereafter also provides protection from exposure to solar UV radiation. Each formulation, especially the Example 2.7 formula, is applied topically as needed to the skin prior to, during, and/or after exposure to solar radiation or sunlight, and thereafter retards, prevents, or treats the skin from the harmful effects of such solar radiation or sunlight exposure.

### Example 3

The topical skin care compositions of the present invention are formulated into a variety of other topical skin care products to provide additional UV protection as described herein, wherein the topical skin care composition has an aqueous phase in which the ADPR is dissolved or otherwise dispersed. Such products are easily formulated by conventional formulation or manufacturing methods directed to the particular product form, wherein the ADPR is added into the formulation as a relatively heat-stable, water-soluble active for dissolution or dispersion into the aqueous phase of the topical skin care product. Examples of topical skin care products to which ADPR is added for the intended UV protection benefit include the following:

| | Ex. 3.10-3.20 | Ex. 3.21-3.31 | Ex. 3.32-3.42 |
|---|---|---|---|
| | ADPR | ADPR | ADPR |
| Product | (wt/wt %) | (wt/wt %) | (wt/wt %) |
| Insect repellants | 0.0005 to 50 | 0.001 to 10% | 0.01 to 3% |
| Color cosmetics | 0.0005 to 50 | 0.001 to 10% | 0.01 to 3% |
| Lip balms | 0.0005 to 50 | 0.001 to 10% | 0.01 to 3% |
| Moisturizing creams | 0.0005 to 50 | 0.001 to 10% | 0.01 to 3% |
| and lotions | | | |
| Fine perfumes and | 0.0005 to 50 | 0.001 to 10% | 0.01 to 3% |
| aftershaves | | | |
| Hair sprays | 0.0005 to 50 | 0.001 to 10% | 0.01 to 3% |
| Hair mousse | 0.0005 to 50 | 0.001 to 10% | 0.01 to 3% |
| Hair conditioners | 0.0005 to 50 | 0.001 to 10% | 0.01 to 3% |
| Topical anesthetics | 0.0005 to 50 | 0.001 to 10% | 0.01 to 3% |
| Topical pump spray | 0.0005 to 50 | 0.001 to 10% | 0.01 to 3% |
| anesthetics | | | |
| Antibiotic creams/ | 0.0005 to 50 | 0.001 to 10% | 0.01 to 3% |
| ointments | | | |

### Experiment

The purpose of the following experiment was to identify which nucleosides or nucleoside derivatives, if any, can protect the skin from solar UV-A, UV-B, and UV-AB (sunlight) irradiation, as demonstrated by the i) cytoprotective effect against hydrogen peroxide induced cytotoxicity, and ii) PARP inhibition. As shown below, only ADPR as evaluated under the test conditions described hereinafter provided the requisite cytoprotection against hydrogen peroxide and PARP inhibition, and was thus selected for evaluation of the protection from UV solar radiation. Tested materials included AMP, adenosine, ADP-ribose, and ADP-glucose.

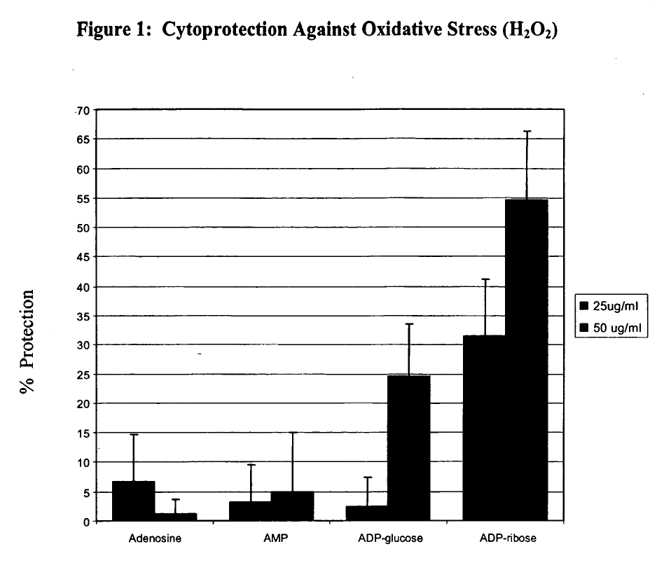

The following describes three experiments that gave rise to the discovery on which the compositions and methods of the present invention are largely based. The first experiment evaluates the ability of Human A549 cells to resist oxidative stress when exposed to peroxide in the presence of selected test materials, e.g. nucleotide derivatives. Summarized results from this experiment are illustrated in the bar chart set forth in FIG. 1.

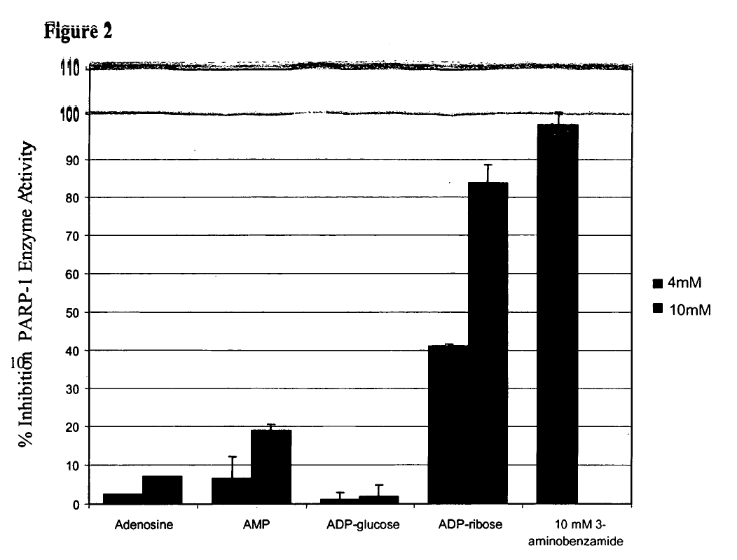

The second experiment evaluates the ability of each test material to provide measurable PARP inhibition. Summarized results from this experiment are illustrated in the bar chart set forth in FIG. 2.

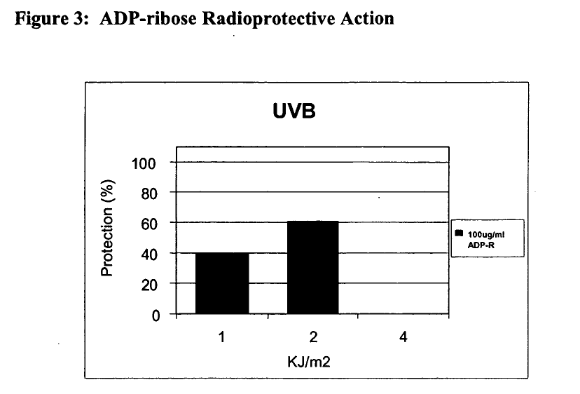

The third experiment evaluates the UV protection provided by the application of ADPR solutions to human immortalized keratinocytes HaCaT cells. Summarized results from this experiment are illustrated in the bar charts set forth in FIGS. 3-5.

### Experiment 1: H₂O₂ Mediated Cytotoxicity

In this first experiment, Human A549 cells are maintained in Dulbeco's modified Eagles medium supplemented with 10% fetal bovine serum, penicillin, streptomycin, and glutamine. The cells are grown up to 60-70% confluency in 24-well plate at 37° C. and 5% CO₂. The cells are incubated in the absence or presence of indicated concentrations of different nucleotide sugars for 2 hours, after which they are treated with 1 mM H₂O₂ along with the nucleotide sugars for an additional 15 hours. Quadruplicate wells are harvested and supernatant pooled for adenylate kinase activity.

A ToxiLight BioAssay Kit from Cambrex is a bioluminescent, non-destructive cytolysis assay kit designed to measure the release of the enzyme, adenylate kinase (AK), from damaged cells. AK is a robust protein present in all eukaryotic cells, which is released into the culture medium when cells die. The enzyme actively phosphorylates ADP (5′-adenosine diphosphate) to form ATP (5′-adenosine triphosphate) and the resultant ATP is then measured using the bioluminescent firefly luciferase reaction. As the level of cytolysis increases, the amount of AK in the supernatant also increases, which results in emission of higher light intensity by the ToxiLight reagent. Because the ToxiLight BioAssay Kit exploits the fact that AK is released from cells when they die, there is no need for a cell lysis step during analysis.

The reaction involves two steps. The first involves the addition of ADP as a substrate for AK. In the presence of the enzyme, AK, the ADP is converted to ATP for assay by bioluminescence: 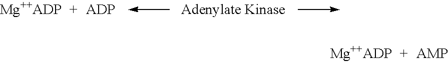
The bioluminescent method utilizes an enzyme luciferase, which catalyzes the formation of light from ATP and luciferin according to the following reaction:

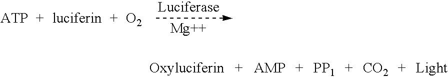

A total volume of 50 μL of sample and 50 μL of the untreated sample are dispensed into each of 3 wells in the microtiter plate Luminometer is primed with injections of reconstituted adenylate kinase detection reagent using the Winglow Software, version 1.25 to control the luminometer. To each well, 100 μL of adenylate kinase detection reagent is added using the injector of the luminometer and incubated 5 minutes. The average RLU (Relative Luciferase Unit) values for each set of triplicate wells are calculated using Winglow software and Microsoft Excel. The formula used to calculate the % inhibition of cytotoxicity is shown below:

$$\frac{( Control mean blanked RLU ) - ( Sample mean blanked RLU )}{( Control mean blanked RLU )} \times 100$$

### Experiment 2: PARP Assay

In this second experiment, each of the test materials is evaluated for its ability to demonstrate measurable PARP inhibition performance.

A. Plate Coating

1. Aliquot 50 μL of the diluted histones into each well.
For 8 samples (make enough for 9)=135 μL histones+1215 μL 1×PBS (Phosphate Buffer Saline)

2. Cover the plate with an adhesive plate sealer and incubate for 2 hours at room temperature (or overnight at 2-8° C.).

B. Plate Blocking

1. Wash plates 4 times with 1×PBS. After the last wash, remove any remaining PBS by decanting. Invert the plate and blot it against clean paper toweling. Note, at this time plates can be air dried and stored covered at room temperature for later use.

2. Block the wells by adding 50 μL of Strep Diluent to each well.

3. Cover the plate with an adhesive plate sealer and incubate for 1 hour at room temperature (or overnight at 2-8° C.).

C. Ribosylation Reaction

Note, do not premix the PARP enzyme and the 2× PARP Cocktail. PARP will autoribosylate in the presence of NAD.

1. Wash plates 4 times with 1×PBS. After the last wash, remove any remaining PBS by decanting. Invert the plate and blot it against clean paper toweling.

2. Add 25 μL of 2× PARP (poly ADP-ribose polymerase) Cocktail to each well.

3. Add inhibitor of interest. The final reaction volume is 50 μL/well.

4. Enzyme is added last.
For 24 wells: Make for 20=2×0.5 units=10 units
20 wells×5 μL=100 μL

10 μL enzyme+90 μl of 1× PARP buffer

| | | | | Diluted |
|---|---|---|---|---|
| Sample | 2X PARP | | | PARP |
| # | Cocktail | Water | Testing compound | enzyme |
| 1 | 25 μL | 25 μL | — | — |
| 2 | 25 μL | 20 μL | — | 5 μL |
| 3 | 25 μL | 17.5 μL | 10 mM 3 AB (amino | 5 μL |
| | | | benzamide) (2.5 μl) | |
| 4 | 25 μL | 12 μL | 4 mM Compound (8 μl) | 5 μL |
| 5 | 25 μL | 4 μL | 8 mM Compound (16 μl) | 5 μL |
| 6 | 25 μL | — | 10 mM Compound (20 μl) | 5 μL |

5. Incubate the plate for 30 minutes at room temperature. The extent of ribosylation is time dependant; therefore this step can be extended if desired.

D. Detection

1. Wash plates 4 times with 1×PBS. After the last wash, remove any remaining PBS by decanting. Invert the plate and blot it against clean paper toweling.

2. Dilute Strep-HRP
Dilute 1:500
   For 20 wells=1,100 μL Strep diluent+2.2 μL Strep-HRP
   Add 50 μL of diluted Strep-HRP to each well.

3. Incubate for 30 minutes at 37° C.

4. Wash plates 4 times with 1×PBS. After the last wash, remove any remaining PBS by decanting. Invert the plate and blot it against clean paper toweling.

5. Add 50 μL TACS-Sapphire dye to each well. Incubate for 10 minutes in the dark. The reaction can be stopped by adding 50 μL of 0.2 N HCl to each well, which will change the color to yellow and be stable for up to 1 hour, and the absorbance read at 450 nm.

### Experiment 3: Protection of Keratinocyte from UV Exposure

In this third experiment, human immortalized keratinocytes HaCaT cells are assessed for the effect of ADP-ribose on UV-induced cell death and its probable mode of action. Here we report that ADP-ribose is able to protect skin cells from exposure to UVA, UVB, and their combination (sunlight).

The spontaneously immortalized human keratinocyte cell line HaCaT contains p53 mutation in both alleles. The cells are cultured in DMEM (Dulbeco's Minimal Essential Medium, Life Technologies) with 10% fetal bovine serum at 37° C. and 5% CO₂. For irradiation, cells are cultured in 100 mm petri dishes to subconfluence (exponential growth), washed with 0.1% EDTA/PBS, and irradiated uncovered as a monolayer.

Treatment 1: 100 μg/ml ADP-R for exposures of 0, 1, 2, and 4 KJ/m² Kodacel filtered UV-B measured with UVB probe. Light source Westinghouse FS-20 bulbs.

Treatment 2: 100 μg/ml ADP-R for exposures of 0, 30, 100 and 300 KJ/m² Kodacel filtered UV-A measured with UVA probe. Lightsource Cosmolux UVA bulbs.

Treatment 3: 100 μg/ml ADP-R for exposures of 0, 150, 450, 600 and 900 J/m² Kodacel filtered solar simulator light measured with a UVB probe. Light source LS1000-8R-AM/LS1000 solar simulator (Solar Light Company) with a 1 kW xenon lamp. This simulator exposure also provides 450, 1350, 1800, and 2700 J/m² UVA, respectively.

Triplicate plates are harvested after 16 hours of incubation and supernatant pooled for adenylate kinase release activity. Sunmarized results from Experiment 3 are illustrated in the bar charts as set forth in FIGS. 3-5.

Results

In the peroxide mediated cytotoxicity experiment, ADP-ribose was evaluated for its ability to protect cells against oxidative stress induced cytotoxicity. To accomplish this, we performed a cytoprotection assay (H₂O₂ oxidation) on human lung epithelial cells as described in the methodology. As shown by the results illustrated in FIG. 1, ADP-ribose at either 25 or 50 μg/ml concentration can protect 30-55% of cells from the cytotoxic effect of H₂O₂.

Moreover, cytoprotection was specific to ADP-ribose when compared to the same concentration of either adenosine, or AMP or ADP-glucose. At higher concentration i.e., 50 μg/ml, ADP-glucose showed some protection (˜25% compared to 55-60% by ADP-ribose) indicating that ADP-glucose might possess similar biological property as ADP-ribose. However, the results shown in FIG. 2 (PARP inhibition study results) ruled out this possibility since ADP-glucose was unable to inhibit PARP activity.

The results summarized in FIG. 2 suggest that ADP-ribose can inhibit 40-80% of PARP activity at a concentration ranging from 4 to 10 mM. Surprisingly, this inhibition was highly specific to the ADP-ribose molecule since the related compounds such as adenosine, AMP, and ADP-glucose were unable to inhibit PARP activity at the same concentration. Moreover, a separate cADP-ribose sample was evaluated and it too was unable to protect cells from cytotoxicity mediated cell death, unlike the structurally similar ADP-ribose material.

Based upon the results of the above-noted experiments, ADP-ribose was tested for its ability to protect skin cells from UV irradiation. The results shown in FIG. 3 indicate that ADP-ribose appears to protect human kerationocytes from UVB rays up to 2 KJ/m² of exposure. The protection is measured in terms of inhibition of cytotoxicity induced by UVB exposure. In UVB mediated cytotoxicity assay in HaCaT cells as shown in FIG. 3, a 40-60% protection of cells by ADP-ribose at 100 ug/ml concentration is observed. It is important to note that UVB present in sunlight is at the range between 1.5 to 2.0 KJ/m². Therefore, a significant protection of skin can be achieved by ADP-ribose against UVB rays present in sunlight.

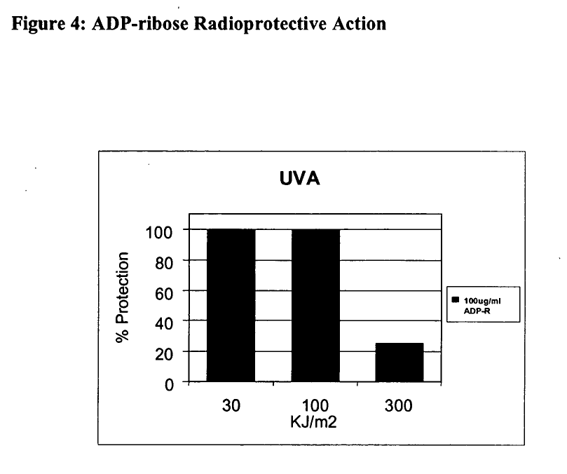

In a related experiment, various UVA exposures indicate that ADP-ribose could also protect 25% of HaCaT cells from the toxicity due to the exposure of UVA at 300 KJ/m² (FIG. 4). This inhibition is quite significant at the level of exposure used i.e. 300 KJ/m². It is now believed that even greater protection could be achieved at lower exposure i.e. <300 KJ/m² of UVA.

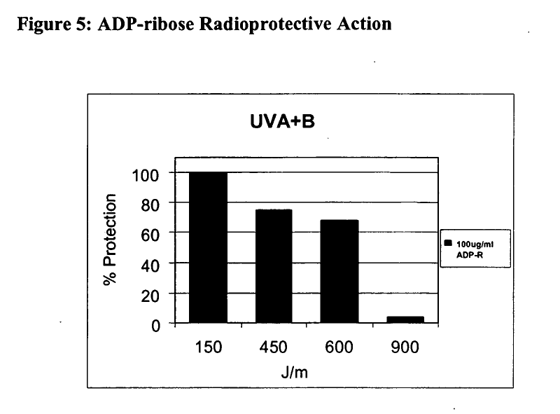

In a related experiment, and as shown in FIG. 5, ADP-ribose at 100 μg/ml protected the human skin cells from a combination of UVA and UVB exposure (solar radiation). In this experiment, significant protection of HaCaT cells was observed up to the exposure of 600 J/m² UVB+1800 J/m² UVA. The 60-70% inhibition of cytotoxicity at 600 J/m² exposure UVB+1800 J/m² UVA is extremely encouraging and so it is now believed that one can expect a significant protection of skin by ADP-ribose at even higher levels of exposure.

1. Skin care compositions comprising 05′-adenosine-diphosphate ribose, and a dermatologically acceptable carrier for the adenosine diphosphate ribose, wherein the composition is applied topically to skin.

2. The skin care composition of claim 1, wherein the composition comprises from about 0.0005% to about 15% by weight of the 05′-adensosine-diphosphate ribose.

3. The skin care composition of claim 1 wherein the composition comprises from about 0.01% to about 3% by weight of the 05′-adenosine-diphosphate ribose.

4. The skin care composition of claim 1 wherein the composition further comprises from about 0.001% to about 30%, by weight, of an additional skin care active selected from the group consisting of desquamatory actives, anti-acne actives, wrinkle repair actives, vitamin B₃ compounds, retinoids, anti-oxidants, radical scavengers, chelators, anti-inflammatory agents, topical anesthetics, tanning actives, skin lightening agents, anti-cellulite agents, flavonoids, antimicrobial actives, antifungal actives, sunscreen actives, conditioning agents, and combinations thereof.

5. The skin care composition of claim 1 wherein the composition is an emulsion.

6. The skin care composition of claim 5 wherein the emulsion is selected from the group consisting of water-in-oil emulsions, oil-in-water emulsions, water-in-silicone emulsions, and combinations thereof.

7. The skin care composition of claim 1 wherein the composition is a rinse-off composition.

8. The skin care composition of claim 1 wherein the composition is a leave-on composition.

9. The skin care composition of claim 8 wherein the leave-on composition is a topical cosmetic.

10. The skin care composition of claim 8 wherein the leave on composition further comprises a skin moisturizer.

11. The skin care composition of claim 8 wherein the leave-on composition further comprises a sunscreen active.

12. The skin care composition of claim 8 wherein the composition is applied topically to the lips.

13. The skin care composition of claim 7 wherein the rinse-off composition further comprises a cleansing surfactant.

14. The skin care composition of claim 1 wherein the composition further comprises a solid, water-insoluble wipe matrix.

15. The skin care composition of claim 1 further comprising a vitamin B₆ compound selected from the group consisting of pyridoxine, pyridoxine esters, amines of pyridoxine, pyridoxine salts, and combinations thereof.

16. The skin care composition of claim 1 wherein the composition further comprises from about 0.1% to about 2.5% pyridoxine HCl.

17. The skin care composition of claim 1 wherein the composition further comprises vitamin A.

18. The skin care composition of claim 1 wherein the composition further comprises vitamin E.

19. The skin care composition of claim 1 wherein the composition further comprises vitamin B₃, vitamin B₅ and vitamin A.

20. A method of preventing, retarding, and/or treating the harmful effects of UV solar radiation on skin, said method comprising the step of applying to skin in need of such treatment a skin care composition comprising 05′-adenosine-diphosphate ribose, and a dermatologically acceptable carrier.

21. The method of claim 20 wherein the skin care composition comprises from about 0.0005% to about 15% by weight of the 05′-adensosine-diphosphate ribose.

22. The method of claim 20 wherein the skin care composition comprises from about 0.01% to about 3% by weight of the 05′-adenosine-diphospphate ribose.

23. The method of claim 20 wherein the skin care composition further comprises about 0.001% to about 30%, by weight, of an additional skin care active selected from the group consisting of desquamatory actives, anti-acne actives, wrinkle repair actives, vitamin B₃ compounds, retinoids, anti-oxidants, radical scavengers, chelators, anti-inflammatory agents, topical anesthetics, tanning actives, skin lightening agents, anti-cellulite agents, flavonoids, antimicrobial actives, antifungal actives, sunscreen actives, conditioning agents, and combinations thereof.

26. The method of claim 20 wherein the method further comprises the step of rinsing or wiping the skin following application.

27. The method of claim 20 wherein the skin care composition is a leave-on composition

28. The method of claim 20 wherein composition is a topical cosmetic.

29. The method of claim 20 wherein the composition further comprises a skin moisturizer.

30. The method of claim 20 wherein the composition further comprises a sunscreen active.

31. The method of claim 20 wherein the skin care composition is applied topically to the lips.

32. The method of claim 20 wherein the skin care composition is a rinse-off composition further comprises a cleansing surfactant.

33. The method of claim 20 wherein the skin care composition further comprises a vitamin B6 compound selected from the group consisting of pyridoxine, pyridoxine esters, amines of pyridoxine, pyridoxine salts, and derivatives thereof.

34. The method of claim 20 wherein the skin care composition further comprises vitamin A.

35. The method of claim 20 wherein the skin care composition further comprises vitamin E.

36. The method of claim 20 wherein the skin care composition further comprises vitamin B₃, vitamin B₅ and vitamin A.

37. A method of preventing, retarding, and/or treating the harmful effects of UV radiation or sunlight on the hair or scalp, said method comprising the step of applying to the hair or scalp a composition comprising 05′-adenosine-diphosphate ribose, and a dermatologically acceptable carrier.

38. The method of claim 37 wherein the composition is a rirse-off shampoo composition.

39. The method of claim 20 wherein the harmful effects of UV solar radiation are selected from the group consisting of photoaging, edema, lympocytic or neturophilic infiltration of the dermis, vasodilation, and dyskeratotic keratinocytes and spongiosis of the epidermis.

40. The method of claim 20 wherein the method is directed to the prevention of skin atrophy or wrinkles.

41. The method of claim 20 wherein the method is directed to the prevention of skin cancer.
